(19) United States
(12) Patent Application Publication
(10) Pub. No.: US 2014/0114142 A1
(43) Pub. Date: Apr. 24, 2014

(54) INTELLIGENT PATIENT MONITOR PENDANT

(71) Applicant: Emanuel Shaoulian, Corona Del Mar, CA (US)

(72) Inventor: Emanuel Shaoulian, Corona Del Mar, CA (US)

(21) Appl. No.: 13/801,159

(22) Filed: Mar. 13, 2013

Related U.S. Application Data
(60) Provisional application No. 61/716,262, filed on Oct. 19, 2012.

(51) Int. Cl. A61B 5/00; A61B 5/0205

(52) U.S. Cl. CPC A61B 5/0006; A61B 5/02055
USPC 600/301; 600/382; 600/384; 607/44

(57) ABSTRACT

A patient monitor system includes a pendant configured to be worn around a neck of a patient. The pendant includes an electrode to sense transthoracic electrical activity and a processor in communication with the electrode. The processor analyzes the transthoracic electrical activity to detect a cardiac event. The pendant also includes a position sensor to determine an orientation of the pendant so as to detect whether the patient is in a horizontal or upright position. The processor determines the accuracy of the detected cardiac event based on data from the position sensor. The patient monitor system may also include additional electrodes connected to the pendant through a necklace. The pendant may also include a verbal verification module to execute verbal communication routines that automatically detect an ability of the patient to communicate verbally so as to further determine the accuracy of the detected cardiac event.

### RELATED APPLICATION

This application claims the benefit under 35 U.S.C. §119(e) of U.S. Provisional Application No. 61/716,262, filed Oct. 19, 2012, which is hereby incorporated by reference herein in its entirety.

### TECHNICAL FIELD

This disclosure is related to patient monitoring. In particular, this disclosure is related to sensing and responding to biological parameters.

### BRIEF DESCRIPTION OF THE DRAWINGS

FIG. 1 illustrates an intelligent pendant worn around a neck of a patient according to one embodiment.

FIG. 2 is a block diagram of the intelligent pendant according to one embodiment.

FIG. 3 is a schematic diagram illustrating inputs to a cardiac event analysis according to one embodiment.

FIG. 4 is a flow chart of a method for monitoring a patient according to one embodiment.

### DETAILED DESCRIPTION OF PREFERRED EMBODIMENTS

Heart disease is the leading cause of death in the United States. A heart attack (also known as an acute myocardial infarction (AMI)) typically results from a thrombus that obstructs blood flow in one or more coronary arteries. AMI is a common and life-threatening complication of coronary heart disease. The sooner that perfusion of the myocardium is restored (e.g., with injection of a thrombolytic medication or with angioplasty), the better the prognosis and survival of the patient from the heart attack. The extent of damage to the myocardium is strongly dependent upon the length of time prior to restoration of blood flow to the heart muscle.

Myocardial ischemia is caused by a temporary imbalance of blood (oxygen) supply and demand in the heart muscle. It is typically provoked by physical activity or other causes of increased heart rate when one or more of the coronary arteries are obstructed by atherosclerosis. Patients will often (but not always) experience chest discomfort (angina) when the heart muscle is experiencing ischemia.

Acute myocardial infarction and ischemia may be detected from a patient's electrocardiogram (ECG) by noting an ST segment shift (e.g., voltage change) over a relatively short (e.g., less than 5 minutes) period of time. Both AMI and ischemia can cause lethal arrhythmias and death if not detected and treated early. Other conditions may also be determined from ECG data. However, approximately 25% of people that have acute myocardial infarction do not feel any symptoms. Thus, certain embodiments disclosed herein measure and analyze ECG data and other patient parameters to detect a patient's condition. Such embodiments may also include methods triggered by the detected patient's condition to communicate with the patient to further assess the patient's condition, to provide instructions to the patient, and/or to provide treatment to the patient. For example, a method may ask a patient whether she/he is feeling chest pains and/or is experiencing a shortness of breath. Based on the patient's condition and/or responses to the queries, the method may instruct the patient to contact her/his doctor or to go to the nearest emergency room. In addition, or in other embodiments, the method may communicate the patient's ECG data to the patient's doctor for further evaluation.

The present disclosure includes an intelligent pendant that may be worn by a patient with heart disease to monitor the patient's current condition, detect cardiac events, verify the detected events through the patient's position and/or through verbal communication with the patient, and communicate with bystanders or remote emergency personnel. Such cardiac events may include, by way of example and not by limitation, ventricular tachycardia (VT or V-tach), ventricular fibrillation, and other cardiac conditions. The pendant may be worn around the patient's neck and includes at least three electrodes and a processor to detect and interpret transthoracic electrical activity of the heart over a period of time. One or more additional electrodes or other biological sensors may be located at other locations on the patient (e.g., in a wristband or in a band worn around the patient's chest, waist, leg, head, or ankle) and may be configured to communicate with the intelligent pendant and/or an external device (e.g., a smart phone or other processing device).

Reference is now made to the figures in which like reference numerals refer to like elements. For clarity, the first digit of a reference numeral indicates the figure number in which the corresponding element is first used. In the following description, numerous specific details are provided for a thorough understanding of the embodiments disclosed herein. However, those skilled in the art will recognize that the embodiments can be practiced without one or more of the specific details, or with other methods, components, or materials. Further, in some cases, well-known structures, materials, or operations are not shown or described in detail in order to avoid obscuring aspects of the invention. Furthermore, the described features, structures, or characteristics may be combined in any suitable manner in one or more embodiments.

Embodiments may include various steps, which may be embodied in machine-executable instructions to be executed by a general-purpose or special-purpose computer (or other electronic device). Alternatively, the steps may be performed by hardware components that include specific logic for performing the steps or by a combination of hardware, software, and/or firmware.

Embodiments may also be provided as a computer program product including a non-transitory, machine-readable medium having stored thereon instructions that may be used to program a computer (or other electronic device) to perform the processes described herein. The machine-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of media/computer-readable medium suitable for storing electronic instructions.

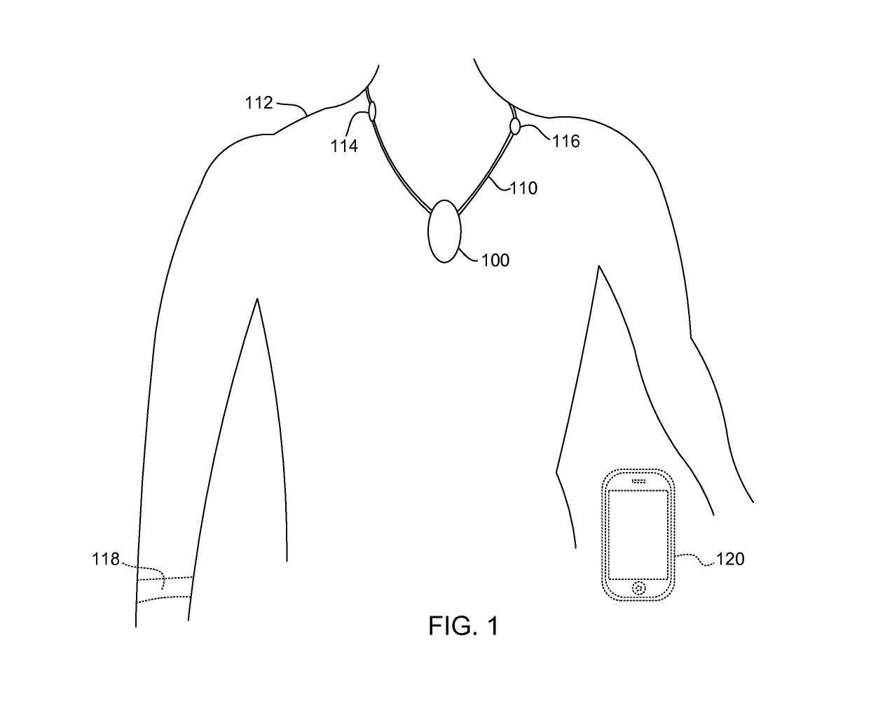

FIG. 1 illustrates an intelligent pendant 100 worn on a necklace 110 around a neck of a patient 112 according to one embodiment. The intelligent pendant 100 includes one or more patient monitoring devices such as electrode(s) to detect electrical signals (corresponding to transthoracic electrical activity) passing along the outer surface of the patient's skin. In this example, the necklace 110 includes at least two additional electrodes 114, 116, which may be positioned at any location along the necklace 110 (including any location along the patient's chest, neck, or back). The necklace 110 may include electrically conductive wires to provide communication between the intelligent pendant and the electrodes 114, 116.

As discussed below, the intelligent pendant 100 may include a processor, memory, and other circuitry to perform the functions described herein. It should be noted that one or more of the devices or elements described as being within or part of the intelligent pendant 100 may be located external to the intelligent pendant 100 in certain embodiments. For example, one or more processors, memories, patient monitors, devices, and/or circuitry may be located in an optional wristband 118, smart phone 120, or other user device. Further, certain elements (e.g., processors and/or memory) may be distributed among the intelligent pendant 100, electrodes 114, 116, wristband 118, smart phone 120, or other user device.

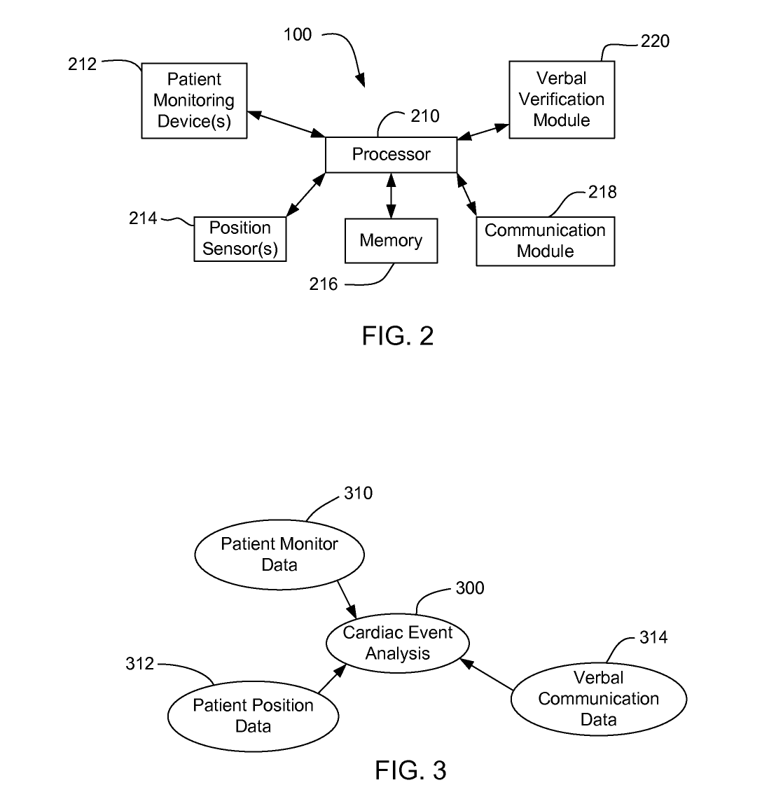

FIG. 2 is a block diagram of the intelligent pendant 100 according to one embodiment. In this example, the intelligent pendant 100 includes a processor 210 in communication with one or more patient monitor devices 212, one or more position sensors 214, a memory device 216, a communication module 218, and a verbal verification module 220. The processor 210 is configured to perform the functions described herein, including analyzing ECG signals to detect cardiac events such as ventricular tachycardia (VT or V-tach), ventricular fibrillation, and sudden cardiac death.

As discussed above, the one or more patient monitoring devices 212 may include electrodes to detect ECG signals. The one or more patient monitoring devices 212 (or any other element on the necklace 110 or wristband 118) may also include other sensors to detect, for example, blood pressure, blood oxygen saturation, pulse rate, temperature, and other biological parameters. In one embodiment, for example, a sensor may be used to detect biological parameters through the patient's skin such as sweat, sweat rates, skin temperature, and blood oxygen saturation. In addition, or in other embodiments, respiration sensor may be used to determine a respiration rate and/breathing patterns. In one embodiment, a wrist band or other device is configured to automatically measure the patient's blood pressure.

The one or more position sensors 214 are configured to determine the patient's position (e.g., standing or lying position). The position sensors 214 may include, for example one or more accelerometer, gyroscope, magnetometer, and/or global positioning satellite (GPS) receiver.

The memory device 216 may include any type of memory for storing computer readable data. The memory device 216 may store, for example, executable instructions for the processor 210 and/or ECG or other sensor data.

The communication module 218 is configured to communicate with external devices and/or emergency personnel. For example, the communication module may use WiFi, Bluetooth, or other wireless communication protocols to communicate with the wristband 118, the smart phone 120, and/or other user devices (e.g., a wireless router or internet gateway). In addition, or in other embodiments, the communication module 218 may be configured to communicate directly through a cellular phone network (e.g., 3G or 4G/LTE network). The communication module 218 may communicate ECG data or other sensed biological parameters. The data may be stored, for example, the in the smart phone 120 and/or communicated to a central monitoring station. The communication module 218 may also provide direct communication between the patient 112 (or bystanders) and emergency personnel such as police, emergency medical technicians (EMTs), doctors, and/or hospitals. Communications with emergency personnel may include GPS location information to assist the emergency personnel in quickly locating the patient.

The verbal verification module 220 is configured to execute verbal communication routines to verify the occurrence of a cardiac event. The verbal verification module 220 may include, for example, an audio processor, speaker, audio amplifier, microphone, and speech recognition algorithms.

FIG. 3 is a schematic diagram illustrating inputs to a cardiac event analysis 300 according to one embodiment. As discussed above, patient monitor data 310 (including ECG and other biological parameter data), patient position data 312, and verbal communication data 314 are provided to the cardiac event analysis 300 to determine whether an actual cardiac event has occurred that requires a response, or whether detected signals are false alarms. In other words, interaction with the patient reduces the likelihood of false alarms and increases the likelihood that appropriate assistance is provided when needed and in a timely manner. For example, if the sensors detect a flat ECG signal, the disclosed system can verbally speak to the patient and receive a verbal response from the patient to verify whether the patient is conscious or experiencing any difficulty. As another example, an alarm may be immediately triggered if a certain cardiac event is detected at about the same time that the patient position data 312 indicates a sudden change from a standing position to a lying position.

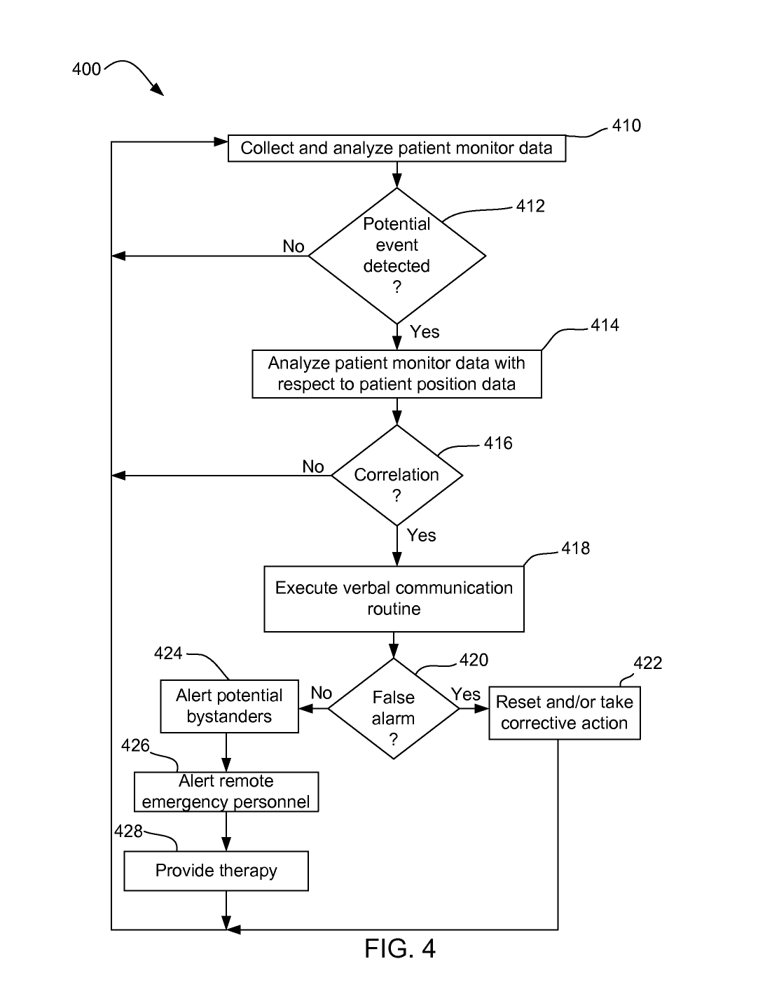

FIG. 4 is a flow chart of a method 400 for monitoring a patient according to one embodiment. The method 400 includes collecting and analyzing 410 patient monitor data and querying 412 or analyzing the patient monitor data to determine whether a potential event is detected. If a potential event has been detected, the method 400 includes analyzing 414 the patient monitor data with respect to patient position data, and querying 416 whether a correlation exists between patient monitor data and the patient position data. If a correlation exists, the method 400 includes executing 418 a verbal communication routine to obtain verbal input from the patient, if possible to validate or invalidate the existence of the event.

Based on at least one of the position data and the verbal communication data, the method determines 420 whether a false alarm condition exists. If a false alarm condition exists, the method resets 422 and/or takes other corrective action (including, e.g., providing verbal instructions to the patient to correct the problem). If a false alarm condition does not exist and the event is determined to be correct, the method 400 includes alerting 424 potential bystanders (e.g., by sounding an alarm or through verbal communication) and/or alerting 426 remote emergency personnel.

In certain embodiments, the method 400 optionally includes providing 428 therapy. The therapy may include, for example, defibrillation and/or pacing. For example, if the detected event includes a cardiac arrhythmia of ventricular fibrillation or ventricular tachycardia in a patient, the intelligent pendant 100 may provide an electrical shock to stop the arrhythmia and allow the heart to reestablish an effective rhythm. As another example, the detected event may be an increase in the patient's physical activity that requires an increase in the artificial base pacing rate, or detection of intrinsic cardiac activity such as atrial and ventricular depolarizations that indicates a need to change the artificial base pacing rate, and the intelligent pendant 100 may respond by providing the pacing according to pacing rate response algorithms. In certain embodiments, the intelligent pendant 100 directly provides therapies such as defibrillation and/or pacing. In other embodiments, the intelligent pendant 100 controls one or more other devices to provide therapies such as defibrillation and/or pacing. For example, the intelligent pendant 100 may control a separate vest or other device worn by the patient that provides external defibrillation and/or pacing.

Skilled persons will recognize from the disclosure herein that the method 400 may be modified based on the particular application. For example, one or more of the steps may be omitted, or the step of executing 418 the verbal communication routine may occur before the step of analyzing 414 the patient monitor data with respect to the patient position data.

It will be understood to those having skill in the art that many changes may be made to the details of the above-described embodiments without departing from the underlying principles of the invention. The scope of the present invention should, therefore, be determined only by the following claims.

1. A patient monitor, comprising:
a pendant configured to be worn around a neck of a patient, the pendant comprising:
a first electrode to sense transthoracic electrical activity;
a processor in communication with the first electrode, the processor to analyze the transthoracic electrical activity to detect a cardiac event; and
a position sensor to determine an orientation of the pendant so as to detect whether the patient is in a horizontal or upright position,
wherein the processor is further configured to determine the accuracy of the detected cardiac event based on data from the position sensor.

2. The patient monitor of claim 1, further comprising:
a second electrode attached to the pendant through a necklace; and
a third electrode attached to the pendant through the necklace,
wherein the second electrode and the third electrode are configured to further sense the transthoracic electrical activity and to communicate respective sensed portions of the transthoracic activity to the processor.

3. The patient monitor of claim 2, further comprising:
a fourth electrode in wireless communication with the processor and configured to further sense the transthoracic electrical activity, the fourth electrode configured to be worn by the patient separate from the necklace.

4. The patient monitor of claim 3, wherein the fourth electrode is integrated with a band configured to be worn by the patient, the band selected from a group comprising a wristband, a chest band, a waist band, a leg band, a head band, and an ankle band.

5. The patient monitor of claim 4, wherein at least one of the pendant, the necklace, and the band further comprises a sensor to detect a biological parameter selected from a group comprising blood pressure, blood oxygen saturation, pulse rate, temperature, skin temperature, sweat, sweat rate, respiration rate, and breathing patterns.

6. The patient monitor of claim 1, wherein the pendant further comprises:
a verbal verification module to execute verbal communication routines that automatically detect an ability of the patient to communicate verbally so as to further determine the accuracy of the detected cardiac event.

7. The patient monitor of claim 6, wherein the verbal verification module comprises:
an audio processor to provide prompts to the patient;
a speaker to provide audible output from the audio processor;
a microphone to detect audio; and
a speech recognition module to determine that the detected audio includes verbal communication from the patient.

8. The patient monitor of claim 1, further comprising a communication module to communicate the detected cardiac event and the determined accuracy to a remote location.

9. The patient monitor of claim 8, wherein the communication module is further configured to communicate at least one of sensed biological parameters to remote medical personnel and global positioning satellite (GPS) data to emergency response personnel.

10. The patient monitor of claim 8, wherein the communication module is further configured to provide verbal communication between remote personnel and the patient or bystanders.

11. The patient monitor of claim 1, wherein the position sensor comprises one or more module selected from the group comprising an accelerometer, a gyroscope, a magnetometer, and a global positioning system (GPS) receiver.

12. A method for monitoring a patient, the method comprising:
collecting and analyzing patient monitor data to detect a potential event;
determining a correlation between the patient monitor data and patient position data;
executing a verbal communication routine that outputs verbal communication data corresponding to an attempted detection of verbal input from the patient;
based on at least one of the patient position data and the verbal communication data, determining whether a false alarm condition exists; and
communicating, to a remote location, an indication of whether the false alarm condition exists.

13. The method of claim 12, further comprising:
providing verbal instructions to the patient to correct the false alarm condition.

14. The method of claim 12, wherein if the false alarm condition does not exist, executing one or more actions selected from a group comprising sounding an alarm, establishing verbal communication with bystanders, and notifying remote emergency personnel.

15. The method of claim 12, wherein at least a portion of the patient monitoring data is received from a pendant worn around the patient's neck.

16. The method of claim 15, wherein if the false alarm condition does not exist, providing treatment to the patient through the pendant.

17. The method of claim 16, wherein the treatment is selected from the group comprising defibrillation and pacing.

18. The method of claim 12, further comprising:
automatically querying the patient, using the verbal communication module, for input corresponding to the patient's condition; and
in response to receive the requested input from the patient, providing at least one of instructions and treatment to the patient.

19. The method of claim 18, wherein automatically querying the patient comprises verbally asking the patient a question related to a group comprising chest pain and shortness of breath.

20. The method of claim 18, further comprising communicating electrocardiogram (ECG) data associate with the patient to a doctor for further evaluation.
